(19) United States
(12) Patent Application Publication
(10) Pub. No.: US 2008/0097459 A1
(43) Pub. Date: Apr. 24, 2008

(54) Device For The Insertion Of Deformable Intra-Ocular Lenses

(75) Inventors: René Kammerlander, Dietikon (CH); Klaus Deinzer, Schlieren (CH)

Correspondence Address: SQUIRE, SANDERS & DEMPSEY L.L.P., 1 MARITIME PLAZA, SUITE 300, SAN FRANCISCO, CA, 94111, US

(73) Assignee: ADVANCED VISION SCIENCE, INC., Goleta, CA (US)

(21) Appl. No.: 11/578,821

(22) PCT Filed: Apr. 22, 2004
(86) PCT No.: PCT/CH04/00242

§ 371 Date: Nov. 19, 2007

(51) Int. Cl. A61F 9/00

(52) U.S. Cl. 606/107

(57) ABSTRACT

The invention relates to a device for inserting deformable intraocular lenses. Said device comprises a housing (1) for receiving an intraocular lens in an elastically deformed state, a cannula (3), and an advancing mechanism (4-10) for transporting the intraocular lens through the cannula (3) into an eye. The injector is suitable for a two-stage working method wherein one person prepares the injector and another person injects the intraocular lens into the eye. Furthermore, the advancing mechanism is embodied in such a way that the intraocular lens can be transported into the cannula (3) by means of a translatory actuating movement on an actuating head (9), and the intraocular lens can be ejected from the cannula (3) by means of a rotatory actuating movement on a pivoting part (10).

### FIELD OF THE INVENTION

The invention relates to a device for the insertion of deformable intra-ocular lenses. The device includes a housing in which an intra-ocular lens in an elastically deformed condition can be accommodated, a cannula, and an advancing mechanism for transporting the intraocular lens through the cannula into an eye. The advancing mechanism comprises a plunger which can be moved in the translational direction and is intended to transport, at its tip, the intra-ocular lens.

### BACKGROUND OF THE INVENTION

Devices for the insertion of deformable intra-ocular lenses are known. In general, their principal purpose consists in being able to keep as small as possible the eye incision necessary for the insertion of an intra-ocular lens.

Known as advancing mechanisms for such devices are, in particular, plungers, which are operated in the manner of a conventional hypodermic syringe for fluids, where the plunger is moved in the translational direction and, with this movement, conveys the intra-ocular lens through the cannula into the eye of the patient. As examples of such injectors, let reference be made here to the documents WO2004/010903A1 or U.S. Pat. No. 6,179,843B1. When using injectors of this type it is felt by many surgeons to be disadvantageous that the movement during the actuation of the injector for transporting the intra-ocular lens through the cannula into the eye takes place in the axial direction of the injector, in the same direction as that in which the cannula has previously been introduced through a small incision into the eye. Because of this, it is difficult to hold the cannula exactly in its position in the eye during the injection. In addition, it is not ruled out that the hand actuating the plunger could slip during the actuation, which could lead to a backward axial movement of the cannula and as a consequence to injuries to the eye.

Injectors have also already been proposed in which a turning actuation is converted into an axial movement of the plunger. Such an injector is mentioned in the European Patent Application EP1332731A1 (corresponding to US2003/139749) as known state of the art. Therein an actuation part mounted at the back on the injector bears the plunger and is provided with an inner thread, which works together with a corresponding outer thread on the housing. In the document it is mentioned as a disadvantage of this injector that two hands are needed for its actuation. In the same document an improved injector is described, which in principle is constructed in the same manner as the above-described injector with turning actuation. In order to give the user the choice of operating the injector with both hands or with only one hand, in this improved injector the thread connection is formed so that it turns if an axial pressure is exerted at the back on the actuator part. Through this measure the improved injector can optionally be operated like a hypodermic syringe with one hand or by a turning actuation like the injector described above.

None of the above-described, known injectors take into account the circumstance that in operations an assistant customarily prepares the instruments, in particular also the injector, and then gives it to the surgeon so that she/he can inject the lens. In the case of an injector for an intra-ocular lens a part of this preparation is the insertion into the injector of the intra-ocular lens required for the engagement, and in particular checking whether the lens is in the correct, compressed, i.e., folded or rolled-up, state and can be pushed through the cannula. This last step is also required in the case of injectors, which are delivered with an intra-ocular lens already laid in, and which are intended for one-time use. Advantageously, the last-named step of the preparation is carried out by the intra-ocular lens being advanced into the cannula. If the cannula consists of a transparent material, it can also be checked visually in that step whether the intra-ocular lens is correctly folded and has assumed the position desired for the injection.

With the injectors known from the state of the art, the two-stage mode of operation described above is difficult in as much as the assistant does not know precisely how far the intra-ocular lens may be advanced into the cannula before the injector is handed to the surgeon. Specifically, as soon as the intra-ocular lens is advanced too far forward in the cannula during the preparation, it can no longer be moved back, since the lens, as mentioned, is advanced by means of a plunger in the cannula. If, during the preparation of the injector, the intra-ocular lens is advanced so far that a part of the lens projects beyond the open end of the cannula, the lens begins to unfold due to its intrinsic elasticity and cannot be injected. Under certain circumstances, due to this not only the intra-ocular lens in question becomes unusable but rather also the injector, specifically when it is an injector intended for one-time use.

### SUMMARY OF THE INVENTION

Proceeding from this state of the art, the invention is based on the objective of forming a device for the insertion of deformable intra-ocular lenses in such a manner that its operation necessarily takes place in two steps so that the device is particularly suitable for a two-step operational process and the above-stated faults in the use of the device cannot occur.

This objective is realized according to the invention by the fact that the advancing mechanism comprises a first actuating element, which can be actuated in the translational direction, and a second actuating element, which can be actuated in the rotational direction, that the first actuating element, which can be actuated in the translational direction, is guided in the housing by a first guide means and in a manner such that it is secure against rotation, that the second actuating element, which can be actuated in the rotational direction, is guided in the housing by a second guide means and in such a manner that it cannot be moved in the translational direction, that the first actuating element, which can be actuated in the translational direction, can be moved from a first position in which it projects out of a component of the device into a second position in which it does not project out of that component, that the first actuating element, which can be actuated in the translational direction, can bring the plunger, starting from a base position in the housing, no further than into an intermediate position in which the tip of the plunger is at a distance from the open end of the cannula, and that the second actuating element, which can be actuated in the rotational direction, can bring the plunger from the intermediate position into a final position, in which the tip of the plunger reaches at least up to the open end of the cannula.

Forming the injector in this way has in particular the advantage that the division of labor between assistant and surgeon also extends to the operation of the injector and incorrect operation is practically ruled out. Due to the fact that two actuating elements are present, faulty manipulations in the preparation of the device are practically ruled out. Due to the fact that the first actuating element, which can be actuated in the translational direction, assumes two positions visible from outside, specifically a first position in which it projects out of a component of the device and a second position in which it does not project out of that component, it can be checked at any time whether the device is prepared for the injection or not.

If, according to a particular type of embodiment of the invention, the cannula is transparent, a visual check of the position of the intra-ocular lens in the cannula is made possible.

According to one type of embodiment of the invention the plunger is guided in the housing in a manner secure against rotation. Guiding the plunger in a manner secure against rotation increases the confidence that the intra-ocular lens can be placed in the correct orientation in the eye because if the plunger were to turn during the advancing movement, due to the frictional contact of the plunger with the intra-ocular lens a torque would be transmitted to the intra-ocular lens in an undesirable manner.

Advantageously, the first actuating element is connected to the plunger in such a manner that it cannot be moved and preferably is connected as one piece with it. Thus, the translational actuating movement can be very simply and directly transferred from the first actuating element to the plunger.

According to another type of embodiment, the first actuating element and the second actuating element comprise threaded parts, which come into engagement with one another when the first actuating element reaches its second position. Thus, a turning actuation of the second actuating element would only be effective after the first actuating element had been actuated in the translational direction.

Advantageously, the first actuating element is accommodated coaxially in the second actuating element and projects beyond it as long as the first actuating element is not in its second position. This mode of construction has the advantage that the first actuating element can, for example, be actuated by pressing with a finger, e.g., the thumb, only until it no longer projects beyond the second actuating element.

An embodiment example of the invention will be described, by way of example, in the following with reference to the accompanying drawings. Shown are:

### BRIEF DESCRIPTION OF THE DRAWINGS

FIG. 1 is a perspective, partially sectioned view of an embodiment example of the injector in the position before its preparation,

FIG. 2 is the injector according to FIG. 1 in the prepared position in which it is handed over to the surgeon, and

FIG. 3 is the injector after the injection of an intra-ocular lens.

### DESCRIPTION OF PREFERRED EMBODIMENTS

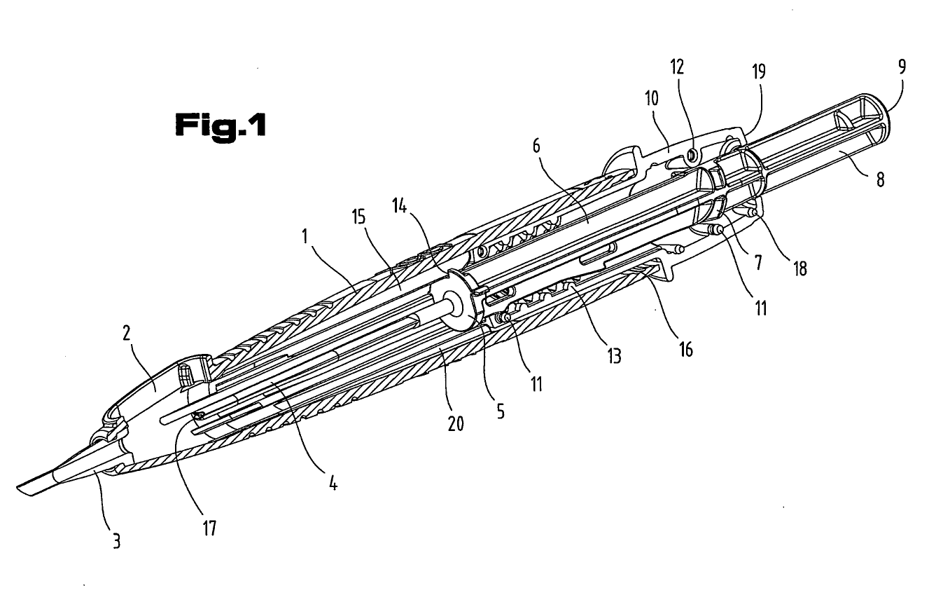

The injector represented in FIGS. 1 to 3 comprises a housing 1 in which the functional elements are disposed. At its front end the injector carries a cannula 3 through which a compressed intra-ocular lens is injected into the eye of a patient. A duct 2 serves to receive a lens holder, which is not represented in the figures because it is not important for the object of the present invention. In the housing 1 a plunger 4 is mounted which has the objective of pushing the intra-ocular lens out of the lens holder first into the cannula 3 and later into the eye of a patient, as is described still more precisely further below. The plunger 4 is, from front to back, connected to a guide part 5, an intermediate part 6, and a sliding part 8 as one piece. On the back end of the sliding part 8 an actuating head 9 is formed.

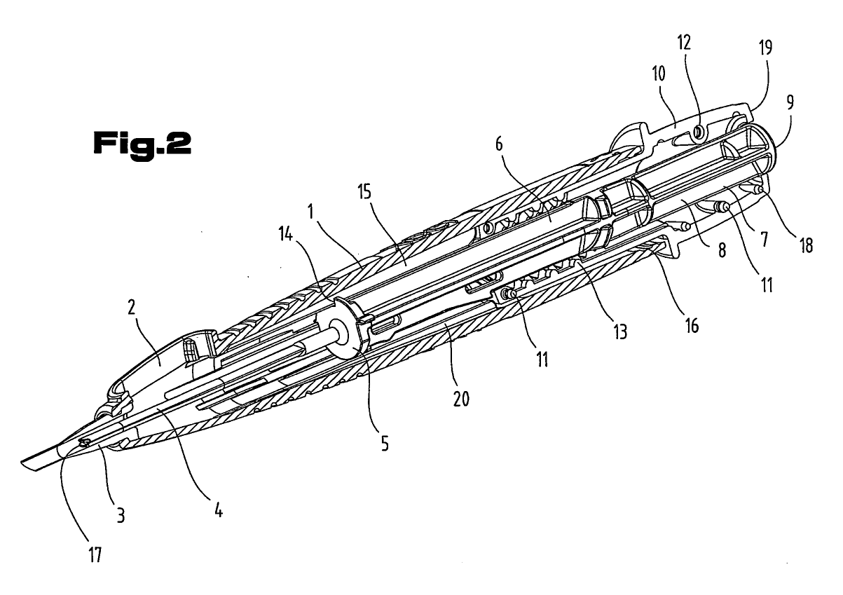

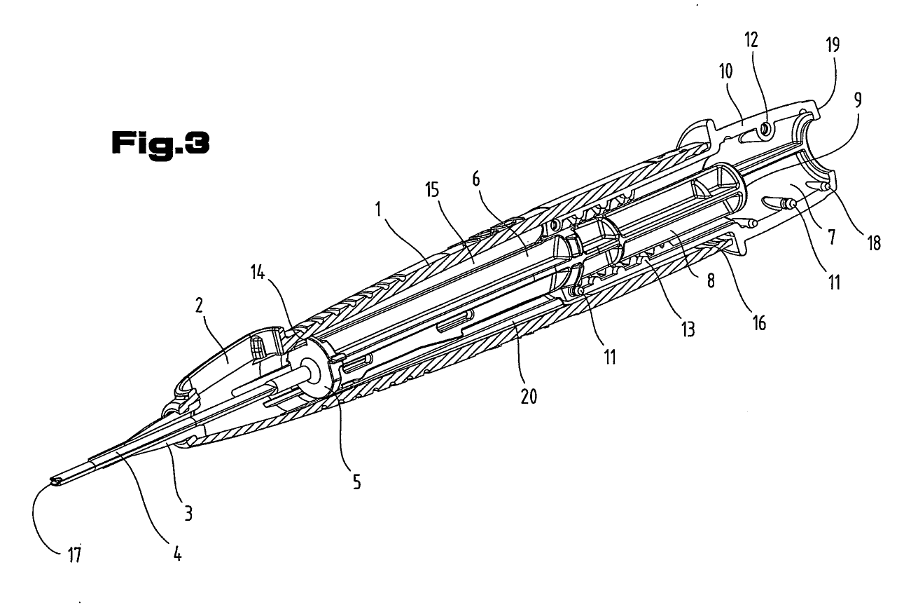

At the back end of the housing 1 a turning part 10 is disposed which in the present example is composed of two shell-like parts which are connected to one another by studs 11, each of which engages in a corresponding hole 12 on the opposite part. Along with this, positioning cams 18 promote the cohesion of the two parts of the turning part 10. The turning part 10 is held in such a manner that it can rotate in the housing, e.g. by its engaging with a projection (not shown) in an annular groove 16 applied at the back end of the housing 10. Adjacent to its front end the turning part 10 has an inner thread 13 whose function is explained in more detail in the description of FIGS. 2 and 3.

In the position represented in FIG. 1 an assistant will therefore insert an intra-ocular lens in the deformed state into the injector with the aid of a lens holder. Thereafter the assistant will operate the actuating head 9 and thereby advance the plunger 4 in the housing 1. Through the translatory actuation movement the plunger tip 17 will come into contact with the intra-ocular lens and push it into the cannula 3. The movement is limited by an outer thread 7 which is disposed on the intermediate part 6 and is on the inner thread 13 of the turning part 10 at the moment at which the actuating head 9 is flush with the back end face 19 of the turning part 10. Here the components of the injector are dimensioned so that in this position the plunger tip 17 is so far in front of the mouth of the cannula 3 that the intra-ocular lens is completely received in the cannula. During said movement the guide part 5 slides on supporting ribs 20 which are disposed in the housing 1. In addition, a guide rib 15 disposed in the housing 1 engages in a slot 14 which is disposed on the guide part 5 and thus prevents turning of the plunger 4, and of the parts connected with it as one piece, relative to the housing 1. At the end of the translatory actuating movement the injector assumes the position represented in FIG. 2.

As can be seen in FIG. 2 the plunger tip 17 projects into the cannula 3 and is at a distance from the mouth of the cannula 3 such that the deformed intra-ocular lens is received completely in the cannula 3. If the cannula 3 is transparent, the assistant can check visually whether the intra-ocular lens sits correctly in the cannula 3. If this is the case, the injector is given to the surgeon for the purpose of injection of the intra-ocular lens.

The surgeon accepts the injector in the position represented in FIG. 2 and guides the tip of the cannula 3 through a small incision into the lens pocket in the eye of the patient in question. Thereafter she/he turns the turning part 10, where the inner thread 13 of the turning part 10 comes into engagement with the outer thread 7 of the intermediate part 6. Since the plunger 4 is secured by the guide rib 15 and the guide part 5 against turning in the housing 1, the turning actuation of the turning part 10 leads to an additional displacement of the plunger in the forward direction so that the intra-ocular lens is ejected from the cannula 3 and is placed in the eye. As can be seen in FIG. 3, the plunger tip 17 projects at the end of the rotational actuating movement over the mouth of the cannula 3 so that the intra-ocular lens is conveyed safely into the eye. There the intra-ocular lens, as a consequence of its elasticity, immediately assumes its original, undeformed form.

The initially described embodiment example of the injector according to the invention is intended for one-time use. From the position represented in FIG. 3 the plunger can in fact be withdrawn by turning the turning part 10 in the opposite direction but only up to the position according to FIG. 2. Since in this position the actuating head 9 does not project out of the turning part 10 and the thread goes out of engagement, the plunger 4 cannot be moved further in the backward direction and thus also a new intra-ocular lens cannot be inserted into the injector.

### LIST OF REFERENCE NUMBERS

1 Housing
2 Duct
3 Cannula
4 Plunger
5 Guide part
6 Intermediate part
7 Outer thread
8 Sliding part
9 Actuating head
10 Turning part
11 Stud
12 Hole
13 Inner thread
14 Slot
15 Guide rib
16 Annular groove
17 Plunger tip
18 Positioning cam
19 End face
20 Supporting rib

1-6. (canceled)

7. A device for the insertion of a deformable intra-ocular lens, comprising:
a housing for accommodating an intra-ocular lens in an elastically deformed condition;
a cannula having an open end; and
an advancing mechanism for transporting the intra-ocular lens through the cannula into an eye, the advancing mechanism comprising
a plunger having a tip, the plunger being moveable in a translational direction and being able to transport the intra-ocular lens with the tip,
a first actuating element being actuatable in the translational direction,
a second actuating element being actuatable in a rotational direction,
wherein the first actuating element is guided in the housing by a first guide mechanism and in a manner such that the first actuating element is secured against rotation,
wherein the second actuating element is guided in the housing by a second guide mechanism and in such a manner that the second actuating element cannot be moved in the translational direction,
wherein the first actuating element is moveable from a first position in which the first actuating element projects out of the second actuating element into a second position in which the first actuating element does not project out of the second actuating element,
wherein the first actuating element is designed to bring the plunger, starting from a base position in the housing, no further than into an intermediate position in which the tip of the plunger is at a distance from the open end of the cannula, and
wherein the second actuating element is designed to bring the plunger from the intermediate position into a final position, in which the tip of the plunger reaches at least up to the open end of the cannula.

8. The device according to claim 7, wherein the cannula is transparent.

9. The device according to claim 8, wherein the plunger is guided in the housing in a manner that the plunger is secured against rotation.

10. The device according to claim 9, wherein the first actuating element is connected to the plunger in such a manner that it cannot be moved relative to the plunger.

11. The device according to claim 10, wherein the first actuating element and the plunger form a single piece.

12. The device according to claim 11, wherein the first actuating element and the second actuating element comprise threaded parts, which come into engagement with one another when the first actuating element reaches its second position.

13. The device according to claim 12, wherein the first actuating element is is accommodated coaxially in the second actuating element and projects beyond the second actuating element as long as the first actuating element is not in its second position.

14. The device according to claim 7, wherein the plunger is guided in the housing in a manner that the plunger is secured against rotation.

15. The device according to claim 7, wherein the first actuating element is connected to the plunger in such a manner that it cannot be moved relative to the plunger.

16. The device according to claim 15, wherein the first actuating element and the plunger form a single piece.

17. The device according to claim 7, wherein the first actuating element and the second actuating element comprise threaded parts, which come into engagement with one another when the first actuating element reaches its second position.

18. The device according to claim 7, wherein the first actuating element is is accommodated coaxially in the second actuating element and projects beyond the second actuating element as long as the first actuating element is not in its second position.
